(19) United States
(12) Patent Application Publication
(10) Pub. No.: US 2023/0240965 A1
(43) Pub. Date: Aug. 3, 2023

(54) TOPICAL INSTANT SMOOTHING AND COLORING COMPOSITIONS FOR THE SKIN

(71) Applicant: True Earth Health Products, LLC, Farmingdale, NY (US)

(72) Inventors: Stefanie Caroline Magda, Boca Raton, FL (US); David Pollock, Boca Raton, FL (US); Jonathan Greenhut, Boca Raton, FL (US); David A. Cohon, Melville, NY (US); Jeanne Borgia, Melville, NY (US)

(73) Assignee: True Earth Health Products, LLC, Farmingdale, NY (US)

(21) Appl. No.: 18/162,479

(22) Filed: Jan. 31, 2023

Related U.S. Application Data
(60) Provisional application No. 63/305,347, filed on Feb. 1, 2022.

(51) Int. Cl. A61K 8/65; A61K 8/73; A61K 8/26; A61K 8/25; A61K 8/34; A61Q 19/08; A61K 8/19

(52) U.S. Cl. CPC A61K 8/65; A61K 8/735; A61K 8/26; A61K 8/73; A61K 8/25; A61K 8/345; A61Q 19/08; A61K 8/19; A61K 2800/43; A61K 2800/47; A61K 2800/95; A61K 2800/524

(57) ABSTRACT

The present invention is directed to topical compositions for smoothing and coloring facial skin, and processes for making and using said compositions.

### CROSS-REFERENCE TO RELATED APPLICATIONS

The present application claims priority to U.S. provisional application Ser. No. 63/305,347, filed Feb. 1, 2022, herein incorporated by reference in its entirety.

### FIELD OF THE INVENTION

The present invention relates to topical compositions for instantly smoothing and coloring skin, and processes for making said compositions.

### BACKGROUND

Several topical, instant anti-wrinkle products available to consumers quickly and temporarily tighten the skin, reducing the appearance of facial wrinkles and undereye swelling. However, a common consumer complaint is that the anti-wrinkle products do not provide color coverage for the skin and are not compatible with other makeup products. When using the anti-wrinkle products, the additional application of a makeup composition to provide color to the skin (for instance foundation, tinted moisturizer, concealer, or other makeup) on top of the anti-wrinkle product breaks the bond created by the anti-wrinkle composition and disrupts the temporary anti-wrinkle effect, or is otherwise incompatible with the anti-wrinkle composition, for instance due to unsightly effects from their combined application. Instant anti-wrinkle products currently available to consumers include Plexaderm Rapid Reduction Serum, Strivectin Hyperlift Eye, Peter Thomas Roth Instant FIRMx Eye, and Jeunesse Instantly Ageless.

Upon discovering that the anti-wrinkle compositions are not compatible with their makeup, consumers may need to purchase and try a wide variety of foundations, tinted moisturizers, concealers, and so forth to find one that may have some compatibility with the anti-wrinkle product they are attempting to pair it with.

US Patent Application No. 2021/0093539 discloses cosmetic compositions to treat or prevent wrinkles, fine lines, skin discoloration, and the like, however, the compositions do not describe an instant anti-wrinkle effect.

The present invention attempts to solve these problems, as well as others.

### SUMMARY OF THE INVENTION

Described herein is a topical composition that, when applied to the skin, provides both instant smoothing effects and provides color coverage would be useful for consumers interested in both features.

The composition and methods are set forth in part in the description which follows, and in part will be obvious from the description, or can be learned by practice of the composition and methods. The advantages of the compositions and methods will be realized and attained by means of the elements and combinations particularly pointed out in the appended claims. It is to be understood that both the foregoing general description and the following detailed description are exemplary and explanatory only and are not restrictive of the composition and methods, as claimed.

### BRIEF DESCRIPTION OF THE DRAWINGS

In the accompanying figures, like elements are identified by like reference numerals among the several preferred embodiments of the present invention.

FIG. 1 represents a series of photographs of an adult woman taken before and then 15 minutes, 1 hour, 2 hours, and 3 hours after the topical application of a composition of the present invention to the face of the subject.

FIG. 2 represents a series of photographs of an adult woman taken before and then 15 minutes, 1 hour, 2 hours, and 3 hours after the topical application of a composition of the present invention to the face of the subject.

FIG. 3 represents a series of photographs of an adult woman taken before and then 15 minutes, 1 hour, 2 hours, 3 hours, and 4 hours after the topical application of a composition of the present invention to the face of the subject.

FIG. 4 represents a series of photographs of an adult man taken before and then several minutes with a blow dryer and taking, 45 minutes, 90 minutes, 135 minutes, and 180 minutes after the topical application of a composition of the present invention to the face of the subject.

### DETAILED DESCRIPTION

The below discussion and definitions are intended to guide understanding but are not intended to be limiting with regard to other disclosures in this application. Throughout this application, references to percentage (%) of compositions of the present invention refers to the % by weight of a given substance to the total weight of the composition being discussed, also signified by “w/w” or “wt/wt”, unless stated otherwise.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, cosmetic, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The present invention is directed to instant coloring and smoothing compositions that, upon topical application to skin, immediately color and smooth the skin for instance by reducing the appearance of wrinkles and reducing swelling such as undereye swelling or “bags”. In an embodiment, the compositions are compatible with other makeup products. In addition, the present invention is directed to methods for preparing these compositions.

In an embodiment, a composition of this invention comprises, consists essentially of, or consists of Purified Water, Propylene Glycol, Titanium Dioxide, Sodium Silicate, Magnesium Aluminum Silicate, Glycerin, Isododecane, Xanthan Gum, Sodium Hyaluronate, Cellulose Gum, Sodium Carboxymethylcellulose, Sodium Polyacrylate, Acetyl Hexapeptide-8, Soluble Collagen, Trimethylsiloxysilicate, CI 77492 (Iron Oxide Yellow), CI 77491 (Iron Oxide Red), CI 77499 (Iron Oxide Black), Phenoxyethanol, Ethylhexylglycerin.

In one embodiment, the composition uses pre-mixed colorants by Kobo, more specifically Kobo GLW75PFSP (for white) GLW45GYSP (for yellow), GLW55GRSP (for red) and GL60GBSP (black), with each being pre-dispersed colorants in water, glycerin, sodium polyacrylate, cellulose gum, to help provide consistent color and less mixing time.

In an embodiment, a composition of this invention is prepared by a process comprising the steps of Charging the tank with the purified water. With both main mixing, side sweep agitation at a minimum of 25-35 RPM and a homogenizer mixer mounted at the bottom of the tank, blending in each colorant blend one at a time and mix until homogenous. Continuing agitation, slowly adding in the each of the following ingredients one at a time, not continuing until each is fully dispersed: Sodium Hyaluronate, Magnesium Aluminum Silicate, Glycerin, Xanthan Gum, Propylene Glycol, Sodium Carboxymethylcellulose, Sodium Silicate, Phenoxyethanol, Ethylhexylglycerin. Continuing agitation and recirculating tank by using a rotary pump to draw from the bottom of the tank and pump the batch back to the top of the tank to provide consistent mixing from top to bottom. Adjusting pH if necessary and no heat is needed.

In another embodiment, the composition of the invention comprising, consisting essentially of, or consisting of water, Hyaluronic Acid, Magnesium Aluminum Silicate, glycerin, xanthan gum, propylene glycol, sodium carboxymethylcellulose, sodium silicate, Phenoxyethanol Ethylhexylglycerin, Trimethylsiloxysilicate, acetyl hexapeptide-8, and soluble collagen in a cream for topical application; wherein the topical composition decreases wrinkles under eye bags or periorbital edema of the face within 15 minutes of topical application of the topical composition. In an embodiment, these components are present in amounts indicated in Tables 4 and 5. The compositions are for topical application to the skin and provide both color coverage and instant smoothing effects. In one embodiment, Hyaluronic Acid is at about 1%/weight of the topical composition, Magnesium Aluminum Silicate is about 2.25%/weight of the topical composition, glycerin is about 0.5%/weight of the topical composition, xanthan gum is about 1%/weight of the topical composition, propylene glycol is about 7%/weight of the topical composition, sodium carboxymethylcellulose is at about 1%/weight of the topical composition, sodium silicate is at about 5.5%/weight of the topical composition, Phenoxyethanol Ethylhexylglycerin is about 1%/weight of the topical composition, Trimethylsiloxysilicate is at about 2%/weight of the topical composition, acetyl hexapeptide-8 is at about 0.5%/weight of the topical composition, and soluble collagen is at about 0.5%/weight of the topical composition.

Other anti-wrinkle products that provide a tightening effect typically work as a film former. The effects of the product vary based on the combination of ingredients used to create that film, as well as plasticizers for flexibility and durability of the film. Some add a bit of cosmetic colorants to help mask the imperfections of the formulation or mask the whitening effects common with other tightening products, however, adding too high a level of colorants can interfere with the film forming and either accentuate any cracks in the tightening effects or even break down the film layer so no effect is realized at all. Additionally, the application of other topicals, more specifically those containing oils or other lipophilic materials, before or after use of the typical tightening product can breakdown that film, resulting in less or no tightening benefits at all.

This topical composition has solved the film forming structure to give more flexibility and durability, providing greater tightening and less whitening effect. Additionally, this invention incorporates a silicone resin and volatile cyclic silicone, whereas once evaporation of the volatile cyclic silicone or cyclomethicone, the film is enhanced, making it more durable and protecting the effects from other incompatible ingredients.

In an embodiment, a composition of the present invention is a cream. In an embodiment, a composition of the present invention is a cosmetic composition or is incorporated into a cosmetic composition such as foundation, concealer, or tinted moisturizer. The characteristics and properties of the cream can also be an opaque gel consistency, or alternatively in a liquid form

The topical composition utilizes silicone, in place of synthetic or natural oils, triglycerides and other lipophylic materials, to provide spreadability of the colorants. The silicone has film-forming properties and is known to provide a level of protection from water and other ingredients. The type of silicone used is a highly cross-linked silicone resin, more specifically Trimethylsiloxysilicate, which adds to the film-forming structure of the invention, provides water-resistance and minimize the breakdown of that film. The present topical composition also incorporates Isododecane as an emollient and a volatile solvent for the higher molecular weight of the silicone used, which then evaporates upon application to the skin.

While the invention can be made without silicones, the spreadability and extended time of firming and tightening benefits, without cracking and whitening, is greatly extended, overcoming many of the negative effects from other previous inventions. Another important factor is the pH of the final product, about 9 to about 13 pH, more preferably about 10.0 to about 12.0.

Different variations in the level and ratios of the cosmetic colorants provide for better skin tone matching, not only in the US, but especially in other regions of the world where some may have lighter or darker skin. Additionally, two versions of this topical compositions can be combined to create a more customized experience. This can be done at the consumer level or with dual chamber packaging or other types of dispensing mechanisms.

Different levels of Hyaluronic Acid/Sodium Hyaluronate can provide different levels of hydration, visibly plumping up the skin from the inside. The molecular weights of the HA also play a role, in this embodiment, we used <100 k daltons.

The levels of glycerin may vary to provide additional flexibility in the film forming effects, as well as to act as solvents to help with the dispersion of colorants.

The level of Titanium Dioxide can be varied to vary the color of the final product, as well as the intensity of the color, since the TiO₂ also creates a background color of canvas, helping the colorants to show more or richer.

Sodium Polyacrylate can be adjusted to improve stability of the product, as well as viscosity.

Preservatives, Phenoxyethanol and Ethylhexylglycerin, are not proprietary and can be substituted as needed for other suitable preservatives.

Other non-colorant adjustments can be varied for obvious reasons, known to anyone skilled in the art.

Without being bound by theory, in an embodiment, a component of a composition may provide more than one function, or in an embodiment, may provide only 1 specified function. In an embodiment, components of the present compositions provide the functions identified in Tables 1a and 1b.

TABLE 1a

| | | Function of | Component |
|---|---|---|---|
| | | component in | amounts |
| Component | Composition | composition | (% w/w) |
| 1 | Water | Skin | 71-74% |
| | | Conditioning | |
| | | Agent | |
| 2 | Propylene Glycol | Solvent | 7 |
| 3 | Titanium Dioxide | Colorant | 0.8-3.0 |
| 4 | Sodium Silicate | Skin | 5.5% (NOTE: It is a 40-44% solution, so |
| | | Conditioning | technically it would be 2.2-2.5) |
| | | Agent | |
| 5 | Magnesium Aluminum | Thickening | 2.2-2.4% |
| | Silicate | Agent | |
| 6 | Glycerin | Humectant | 0.5-1.5% |
| 7 | Isododecane | Volatile Silicone | 0.5-0.9% |
| 8 | Xanthan Gum | Thickening | 0.8-1.2% |
| | | Agent | |
| 9 | Sodium Hyaluronate | Humectant | 1% (this is a 1% solution, meaning it is |
| | | | 0.01% and rest water of specific |
| | | | molecular weights of 100k daltons or |
| | | | less) |
| 10 | Cellulose Gum | Thickening | .05-.50% |
| | | Agent | |
| 11 | Sodium | Thickening | 0.8-1.2% |
| | Carboxymethylcellulose | Agent | |
| 12 | Sodium Polyacrylate | Thickening | 0.3-0.7% |
| | | Agent | |
| 13 | Acetyl Hexapeptide-8 | Skin | 0.5% (this is a 0.01% solution, so <0.01% |
| | | Conditioning | of actual peptide) |
| | | Agent | |
| 14 | Soluble Collagen | Skin | 0.5% (this is a 10% solution, so <0.05%) |
| | | Conditioning | |
| | | Agent | |
| 15 | Trimethylsiloxysilicate | Film Former | 1.0-1.5 |
| 16 | CI 77492 | Colorant | 1.2-1.9% (would expand from 1.0-3.0%) |
| | (Iron Oxide Yellow) | | |
| 17 | CI 77491 | Colorant | 0.04-0.15% |
| | (Iron Oxide Red) | | |
| 18 | CI 77499 | Colorant | 0.05-0.7% |
| | (Iron Oxide Black) | | |
| 19 | Phenoxyethanol | Preservative | 0.9-1% |
| 20 | Ethylhexylglycerin | Preservative | 0.08-0.12% |

TABLE 1b

| | | Function of | Component |
|---|---|---|---|
| | | component in | amounts |
| Component | Composition | composition | (% w/w) |
| 1 | Water | Skin | 66-69% |
| | | Conditioning | |
| | | Agent | |
| 2 | Propanediol | Solvent | 6-8% |
| 3 | Glycerin | Humectant | 1.3-1.7% |
| 4 | Sodium Silicate | Skin | 8-9% |
| | | Conditioning | |
| | | Agent | |
| 5 | Magnesium Aluminum | Thickening | 2-3% |
| | Silicate | Agent | |
| 6 | Titanium Dioxide | Colorant | 1.1-1.7% |
| 7 | Vitis Vinifera (Grape) | Skin | 2-3% |
| | Juice Extract | Conditioning | |
| | | Agent | |
| 8 | Sodium Hyaluronate | Humectant | 2.7-3.3% |
| 9 | Xanthan Gum | Thickening | 0.8-1.2% |
| | | Agent | |
| 10 | Cellulose Gum | Thickening | 1.2-1.6% |
| | | Agent | |
| 11 | Sodium | Thickening | 0.8-1.2% |
| | Carboxymethylcellulose | Agent | |
| 12 | Sodium Polyacrylate | Thickening | 0.3-0.5% |
| | | Agent | |
| 13 | CI 77492 | Colorant | 0.4-1.6% |
| | (Iron Oxide Yellow) | | |
| 14 | CI 77491 | Colorant | 0.04-0.15% |
| | (Iron Oxide Red) | | |
| 15 | CI 77499 | Colorant | 0.02-0.2% |
| | (Iron Oxide Black) | | |
| 16 | Phenoxyethanol | Preservative | 0.9-1% |
| 17 | Ethylhexylglycerin | Preservative | 0.08-0.12% |

Without being bound by theory, in an embodiment, a component of a composition may provide more than one function, or in an embodiment, may provide only 1 specified function. In an embodiment, components of the present compositions provide the functions identified in Tables 1a or 1b.

Component 2. Propylene Glycol, is used as a plasticizer, can be substituted with butylene glycol, pentylene glycol, polyethylene glycol, glycerol, dipropylene glycol, propanediol, triethyl citrate, glycerin, natural ingredients such as propanediol, or a combination thereof

Component 4. Sodium Silicate is the primary film former, other variations can use more or less of this ingredient, as well as secondary film formers, such as Pullulan, Albumin, certain PVP/Polymers and others.

Component 5. Magnesium Aluminum Silicate—an anionic associative polymeric thickener, there are other rheology modifiers

Component 6. Glycerin works as a humectant, as well as a plasticizer and may be substituted or combined with glycols/propanediol as described above in #2

Component 7. Isododecane, since it is volatile, evaporates from the skin—leaving the silicone resin to create a more durable film—there are other volatile materials that could be considered, various cyclomethicones or even cyclomethicone replacements, such as Hemi-Squalane

Components 9, 13, 14—Soluble Collagen, Acetyl Hexapeptide, Sodium Hyaluronate and other skin conditioning agents maybe substituted or increased or decreased to provide different skin conditioning effects, whether botanicals, other peptides or proteins, retinoids, vitamins, trace minerals and many others known to those skilled in the art. Component 11 also could be eliminated, they are not necessary for the invention to work

Components 19, 20—Phenoxyethanol, Ethylhexylglycerin are preservatives and can be substituted with a wide variety of available preservatives on the market

In one embodiment, Sodium Polyacrylate is option and not necessary for this invention

In one embodiment, Sodium Silicate—necessary for film forming/tightening.

In one embodiment, Glycerin/Glycol/Silicate-anionic polymer are necessary to create the film effect/flexibility of the film.

In one embodiment, Trimethylsiloxysilicate is necessary to create the durability to work with the colorants, we have done without it, but this greatly improved it.

In one embodiment, Isodeodecane necessary to improve the performance of the Trimethylsiloxysilicate.

The ranges of components % w/w may be varied above or below at least 20% or between about 5% and about 25% from the ranges identified in Table 3 and described herein.

TABLE 3

| | | Function of | Component |
|---|---|---|---|
| | | component in | amounts |
| Component | Composition | composition | (% w/w) |
| 1 | Water | Skin | 71-74% |
| | | Conditioning | |
| | | Agent | |
| 2 | Propylene Glycol | Solvent | 5-9% |
| 3 | Titanium Dioxide | Colorant | 0.5-5.0% |
| 4 | Sodium Silicate | Skin | 5-10% (of a 40% solution) |
| | | Conditioning | |
| | | Agent | |
| 5 | Magnesium Aluminum | Thickening | 1.5-3.0% |
| | Silicate | Agent | |
| 6 | Glycerin | Humectant | 0.5-10% |
| 7 | Isododecane | Volatile Silicone | 0.5-2.0% |
| 8 | Xanthan Gum | Thickening | 0.5-2.5% |
| | | Agent | |
| 9 | Sodium Hyaluronate | Humectant | .01-50% (of a 1% solution) |
| 10 | Cellulose Gum | Thickening | .05-1.0% |
| | | Agent | |
| 11 | Sodium | Thickening | 0.5-2% |
| | Carboxymethylcellulose | Agent | |
| 12 | Sodium Polyacrylate | Thickening | 0.0-2.0% |
| | | Agent | |
| 13 | Acetyl Hexapeptide-8 | Skin | 0.5% (this is a 0.01% solution, |
| | | Conditioning | so <0.01% of actual peptide) |
| | | Agent | |
| 14 | Soluble Collagen | Skin | 0.5% (this is a 10% solution, |
| | | Conditioning | so <0.05%) |
| | | Agent | |
| 15 | Trimethylsiloxysilicate | Film Former | 1.0-1.5 |
| 16 | CI 77492 | Colorant | .5-5.0% |
| | (Iron Oxide Yellow) | | |
| 17 | CI 77491 | Colorant | 0.01-4.0% |
| | (Iron Oxide Red) | | |
| 18 | CI 77499 | Colorant | 0.05-2.0% |
| | (Iron Oxide Black) | | |
| 19 | Phenoxyethanol | Preservative | 0.7-1.2% |
| 20 | Ethylhexylglycerin | Preservative | 0.1-0.8% |

In one embodiment, Isodeodecane necessary to improve the performance of the Trimethylsiloxysilicate.

In an embodiment, a composition of the present invention does not include Retin-A or similar compositions.

In an embodiment, a composition of the present invention does not include oils or minimal amounts of oils.

“Applying”, “application”, “apply”, and the like according to the present invention refers to contacting (such as spreading) an effective amount of a composition of this invention directly on the (external) surface of a subject's skin. Applying a composition of this invention both smooths the skin and provides color coverage to the area. In an embodiment, an effective amount refers to an amount of composition needed to achieve instant smoothing including reduction of wrinkles or swelling such as periorbital edema, along with color coverage of the surface of the skin to which the composition is applied. In an embodiment, color coverage is visible upon application of a composition of this invention to the skin. In an embodiment, “instant” refers to an about 15 minute period of time or less to smooth the skin. In an embodiment, a composition of the present invention may be applied to a specific portion of the subject's face, such as one or more of the undereye area, the brow area, corner of the eye, the periorbital area (under, beside, and above eye), the forehead, the bridge of the nose, the outer edge of the eye and/or temple for instance to smooth and cover “crow's feet”, near the mouth, near the mouth and cheek including for instance smile lines, the cheekbone, the cheek, the jawline, and any other part of the face. In an embodiment, a composition of the present invention may be applied to part or all of the face or other areas of the body including for instance one or more of the neck, the chest, the bosom, and other areas as desired.

In one embodiment, the composition may be applied to any part of the body or skin, such as the hands, arms, legs, arm pits, and the like.

In an embodiment, “smoothing” the skin, and the like, according to this invention may refer to applying a composition to skin having one or more wrinkles to reduce the appearance of the wrinkles. In an embodiment, a wrinkle is a crease or fold in the skin, for instance formed from age, repeated expressions, smoking or other tobacco use, sun exposure, medical treatments such as radiation therapy, exposure to chemicals that detrimentally affect the skin, squinting, tanning in a sunning bed, stress, or poor sleep. In an embodiment, smoothing the skin according to this invention refers to applying a composition to skin with underlying swelling for instance from edema to reduce the swelling. Such swelling may occur with age, allergies, stress, weight loss, or other causes. In an embodiment, a composition of this invention provides instant smoothing by appearing to reduce or reducing wrinkles, fine lines, crow's feet, under-eye bags, and dark circles.

In an embodiment, color coverage, coloring the skin, and the like refer to the present compositions adding color to the skin for instance to blend the area of application with natural skin tone or color to be provided from makeup, such as foundation, tinted moisturizer, or concealer. In an embodiment, the present compositions may be used as a standalone cosmetic, or may be further covered with other cosmetic “makeup” products. In an embodiment, color coverage according to this invention includes covering dark under eye circles, so the color of the undereye area appears to be similar to other areas of the face. In an embodiment, compositions of the present invention are applied prior to the application of other color compositions, or prior to the application of any other compositions.

In an embodiment, a composition of this invention is intended for topical use on the surface of the skin only. In an embodiment, use of a composition of this invention is on skin comprising an epidermis. In an embodiment, a composition of this invention is not intended for use in the eyes, ears, oral cavity, the anal or vaginal cavity, nasal passages, or other cavity or opening of the body. In an embodiment, a composition of this invention is intended for use on unbroken skin, and not intended for use on broken skin. In an embodiment, a composition of this invention is intended for use on healthy skin, and not for instance skin having a rash or other problem on the skin's surface, or under the surface.

Without being bound by theory, when a composition of the present invention is topically applied to the skin of a subject, the composition interacts with the surface of the skin to smooth and color the surface of the skin.

A “subject” according to the present invention is a human, such as an adult man or adult human. A subject may be referred to as a patient or a consumer or, as in the Examples, a tester. Other mammals may be included as a subject.

The present invention may be further understood in connection with the following Examples and embodiments. The Examples and embodiments described throughout this application are provided to illustrate the invention, and are not intended as limiting. Generally, chemical steps may be performed in any order, unless indicated otherwise.

### EXAMPLES

The testing protocol for Examples 1-4 included a multi-step process and entailed working with external product testers: 3 adult women and 1 adult man. Each product tester was screened and pre-qualified before testing the product. To enter the study, testers were required to have facial skin features that may be seen as imperfections or features a subject may wish to change, such as wrinkles, fine lines, crow's feet, under-eye bags, and/or dark circles. During the test, testers (test subjects) were photographed before and after application of a composition of this invention, typically over the span of 2-4 hours. Testers' faces were clear of makeup when testing the product and when photographs were taken.

The composition applied in Examples 1-4 below consisted of the components described in Table 4 where the ranges of components % w/w may be varied above or below at least 20% or between about 5% and about 25% from the ranges identified in Table 5.

TABLE 4

| Component | Composition | CAS | Activity Level |
|---|---|---|---|
| 1 | Water | 7732-18-5 | 100% |
| 2 | Propylene Glycol | | 100% |
| 3 | Titanium Dioxide | 13463-67-7 | 100% |
| 4 | Sodium Silicate | 1344-09-08 | 44% |
| 5 | Magnesium Aluminum | 1327-43-1 | 100% |
| | Silicate | | |
| 6 | Glycerin | 9067-32-7 | 97% |
| 7 | Isododecane | | 100% |
| 8 | Xanthan Gum | 11138-66-2 | 100% |
| 9 | Sodium Hyaluronate | 9067-32-7 | 1% |
| 10 | Cellulose Gum | 9004-32-4 | 100% |
| 11 | Sodium | 9004-32-4 | 100% |
| | Carboxymethylcellulose | | |
| 12 | Sodium Polyacrylate | 9003-04-07 | 100% |
| 13 | Acetyl Hexapeptide-8 | | 0.01% |
| 14 | Soluble Collagen | | 0.50% |
| 15 | Trimethylsiloxysilicate | | 100% |
| 16 | CI 77492 | 51274-00-1 | 100% |
| | (Iron Oxide Yellow) | | |
| 17 | CI 77491 | 1309-37-9 | 100% |
| | (Iron Oxide Red) | | |
| 18 | CI 77499 | 1317-61-9 | 100% |
| | (Iron Oxide Black) | | |
| 19 | Phenoxyethanol | 122-99-6 | 100% |
| 20 | Ethylhexylglycerin | 70445-33-9 | 100% |

For each table the INCI's are included, the actual chemicals, some are blends, and an SDS for each ingredient used.

Below is a table 5 for the formulation using the tradenames/blends. The ranges of components % w/w may be varied above or below at least 20% or between about 5% and about 25% from the % identified.

TABLE 5
Tight & Tighten - Nude

| Component | Composition | Supplier | % |
|---|---|---|---|
| 1 | Water | | QS to 100 |
| 2 | GLW75PFSP White | Kobo | 3.48 |
| 3 | GLW745GYSP Yellow | Kobo | 2.4 |
| 4 | GLW55GRSP Red | Kobo | 0.48 |
| 5 | GLW60GBSP Black | Kobo | 0.096 |
| 6 | Hyaluronic Acid | Innovative | 1 |
| | 100k daltons | Naturals | |
| 7 | Magnesium Aluminum | Vanderbilt | 2.25 |
| | Silicate | | |
| 8 | Glycerin | Jeen/Custom | 0.5 |
| | | Chemical | |
| 9 | Xanthan Gum | | 1 |
| 10 | Propylene Glycol | Jeen/Custom | 7 |
| | | Chemical | |
| 11 | Sodium | | 1 |
| | Carboxymethylcellulose | | |
| 12 | Sodium Silicate/Grade 40 | | 5.5 |
| 13 | Phenoxyethanol | Euxyl | 1 |
| | Ethylhexylglycerin | | |
| 14 | Trimethylsiloxysilicate | Chemisil/ | 2 |
| | | Custom | |
| | | Chemicals | |
| 15 | Acetyl Hexapeptide-8 | Innovative | 0.5 |
| | | Naturals | |
| 16 | Soluble Collagen | Innovative | 0.5 |
| | | Naturals | |

### Example 1: Application of Composition to a Subject

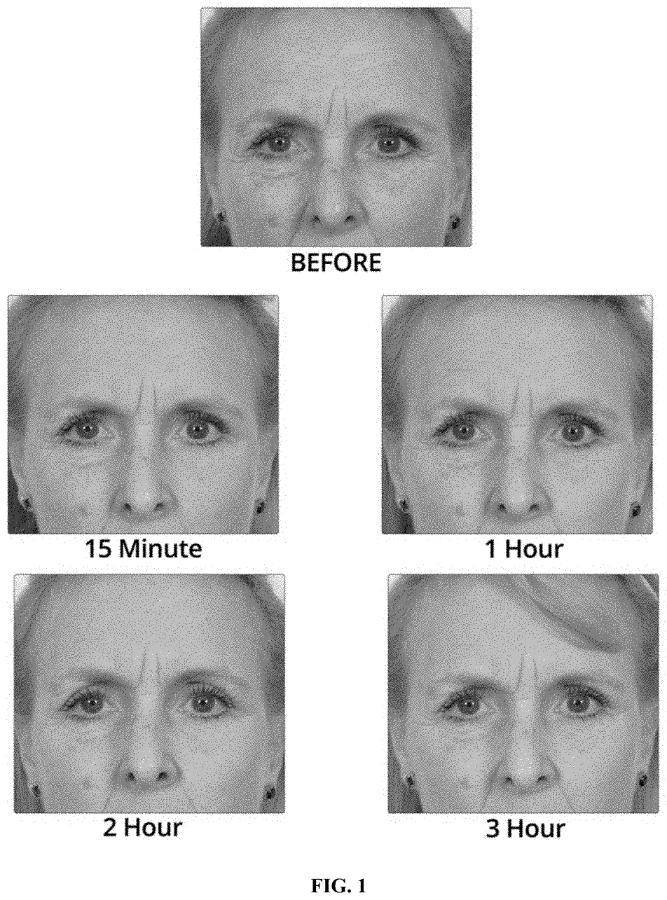

FIG. 1 shows a series of photographs of part of an adult woman's face, taken before topical application of the composition described in Table 5 above and then 15 minutes, 1 hour, 2 hours, and 3 hours after application of the composition. As can be seen from 15 minutes photo, the composition has an instant effect on decreasing wrinkles under eye bags or periorbital edema region of the face, and additional decrease in wrinkles at the 1 hour, 2 hours, 3 hours, and 4 hours after application of the composition.

### Example 2: Application of Composition to a Subject

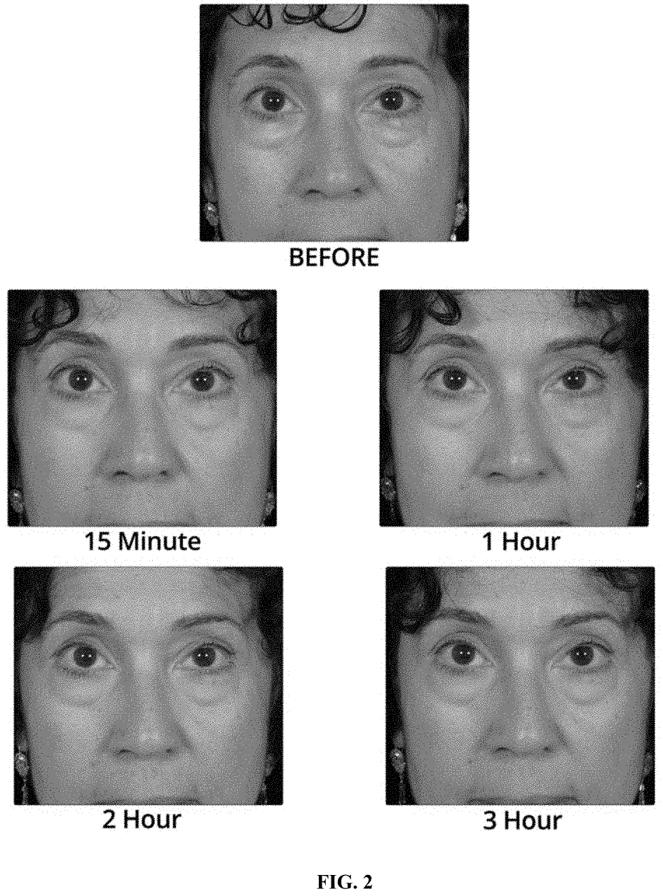

FIG. 2 shows a series of photographs of an adult woman taken before topical application of the composition described in Table 5 above and then 15 minutes, 1 hour, 2 hours, and 3 hours after application of the composition. As can be seen from 15 minutes photo, the composition has an instant effect on decreasing wrinkles under eye bags or periorbital edema region of the face, and additional decrease in wrinkles at the 1 hour, 2 hours, 3 hours, and 4 hours after application of the composition.

### Example 3: Application of Composition to a Subject

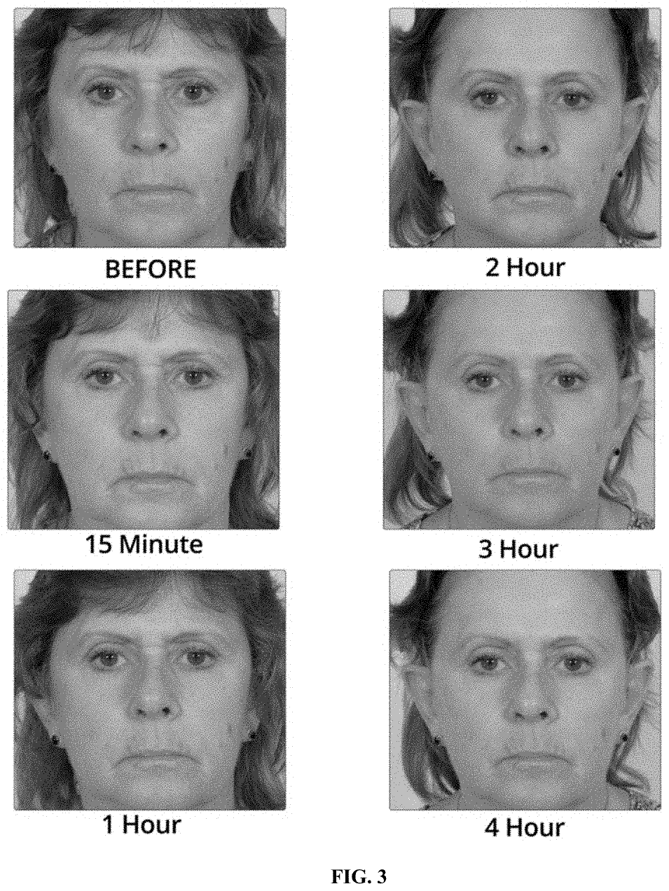

FIG. 3 shows a series of photographs of an adult woman taken before topical application of the composition described in Table 5 above and then 15 minutes, 1 hour, 2 hours, 3 hours, and 4 hours after application of the composition. As can be seen from 15 minutes photo, the composition has an instant effect on decreasing wrinkles under eye bags or periorbital edema region of the face, and additional decrease in wrinkles at the 1 hour, 2 hours, 3 hours, and 4 hours after application of the composition.

### Example 4: Application of Composition to a Subject

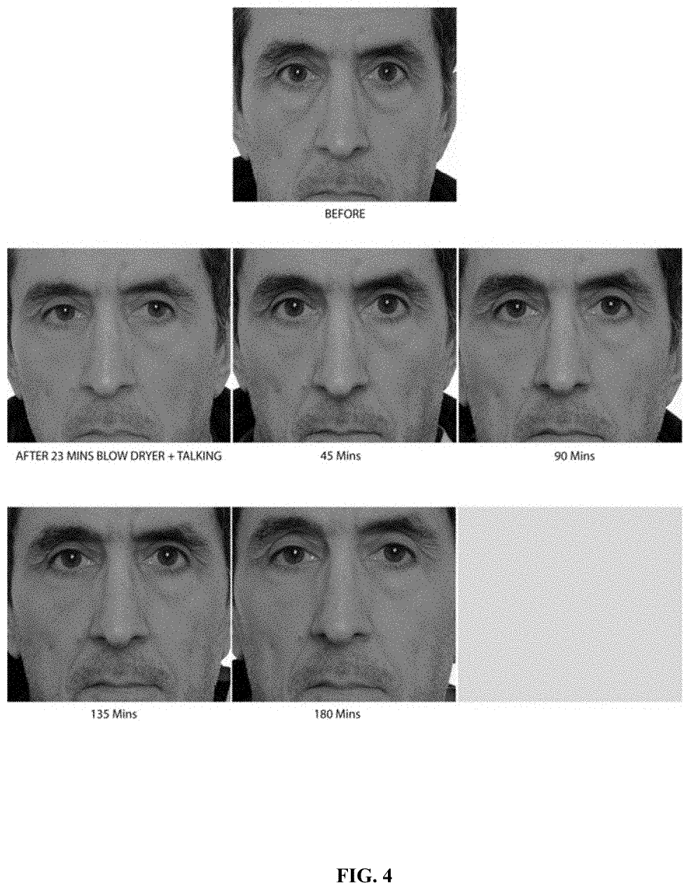

FIG. 4 represents a series of photographs of an adult man taken before and then after treatment with a blow dryer and talking, 45 minutes, 90 minutes, 135 minutes, and 180 minutes after application of the composition described in Table 5. As can be seen from 15 minutes photo, the composition has an instant effect on decreasing wrinkles under eye bags or periorbital edema region of the face, and additional decrease in wrinkles at the 1 hour, 2 hours, 3 hours, and 4 hours after application of the composition.

1. A topical compositions for smoothing and coloring skin comprising: water, Hyaluronic Acid, Magnesium Aluminum Silicate, glycerin, xanthan gum, propylene glycol, sodium carboxymethylcellulose, sodium silicate, Phenoxyethanol Ethylhexylglycerin, Trimethylsiloxysilicate, acetyl hexapeptide-8, soluble collagen in a cream for topical application, and a colorant; wherein the topical composition decreases wrinkles in skin within 15 minutes of topical application of the topical composition and coloring the skin.

2. The topical composition of claim 1, wherein Hyaluronic Acid is about 1%/weight of the topical composition, Magnesium Aluminum Silicate is about 2.25%/weight of the topical composition, glycerin is about 0.5%/weight of the topical composition, xanthan gum is about 1%/weight of the topical composition, propylene glycol is about 7%/weight of the topical composition, sodium carboxymethylcellulose is about 1%/weight of the topical composition, sodium silicate is about 5.5%/weight of the topical composition, Phenoxyethanol Ethylhexylglycerin is about 1%/weight of the topical composition, Trimethylsiloxysilicate is about 2%/weight of the topical composition, acetyl hexapeptide-8 is about 0.5%/weight of the topical composition, soluble collagen is about 0.5%/weight of the topical composition, and the colorant is selected from the group consisting of Iron Oxide Yellow, Iron Oxide Red, and Iron Oxide Black.

3. The topical composition of claim 2, wherein the decrease in wrinkles is increased at 1 hour, 2 hours, and 3 hours after application of the composition.

4. A method of decreasing wrinkles in the skin and coloring facial skin, comprising:
a. Applying a topical composition comprising water, Hyaluronic Acid, Magnesium Aluminum Silicate, glycerin, xanthan gum, propylene glycol, sodium carboxymethylcellulose, sodium silicate, Phenoxyethanol Ethylhexylglycerin, Trimethylsiloxysilicate, acetyl hexapeptide-8, soluble collagen, and a colorant; and
b. Decreasing wrinkles in the skin within 15 minutes of topical application of the topical composition and coloring the skin.

5. The method of claim 4, wherein Hyaluronic Acid is about 1%/weight of the topical composition, Magnesium Aluminum Silicate is about 2.25%/weight of the topical composition, glycerin is about 0.5%/weight of the topical composition, xanthan gum is about 1%/weight of the topical composition, propylene glycol is about 7%/weight of the topical composition, sodium carboxymethylcellulose is at about 1%/weight of the topical composition, sodium silicate is about 5.5%/weight of the topical composition, Phenoxyethanol Ethylhexylglycerin is about 1%/weight of the topical composition, Trimethylsiloxysilicate is about 2%/weight of the topical composition, acetyl hexapeptide-8 is about 0.5%/weight of the topical composition, soluble collagen is about 0.5%/weight of the topical composition, and the colorant is selected from the group consisting of Iron Oxide Yellow, Iron Oxide Red, and Iron Oxide Black.

6. The method of claim 5, further comprising blow drying the topical composition while on the skin of a subject.

7. The method of claim 6, further comprising decreasing wrinkles at the 1 hour, 2 hours, 3 hours, and 4 hours after application of the topical composition.

8. The method of claim 7, further comprising smoothing the skin, and the wrinkle is a crease or fold in the skin formed from age, repeated expressions, smoking or other tobacco use, sun exposure, medical treatments such as radiation therapy, exposure to chemicals that detrimentally affect the skin, squinting, tanning in a sunning bed, stress, or poor sleep.

9. The method of claim 7, further comprising smoothing the skin with underlying swelling for instance from edema to reduce the swelling selected from the group consisting of age, allergies, stress, weight loss, or other causes.

10. The method of claim 9, wherein coloring skin comprises adding color to the skin to blend the area of application with natural skin tone or color to be provided from makeup, including foundation, tinted moisturizer, or concealer.

11. The method of claim 10, wherein coloring the skin includes covering dark under eye circles, so the color of the undereye area is the same as other areas of the face.

12. The method of claim 10, wherein the composition is applied prior to the application of other color compositions, or prior to the application of any other compositions.
